(19) United States
(12) Patent Application Publication
(10) Pub. No.: US 2022/0142970 A1
(43) Pub. Date: May 12, 2022

(54) CANNABIS INHALATION PRODUCTS AND METHODS OF PRODUCTION THEREOF

(71) Applicant: BUZZELET DEVELOPMENT AND TECHNOLOGIES LTD., OR-AKIVA (IL)

(72) Inventors: AHARON EYAL, JERUSALEM (IL); NOA RAZ, GIZO (IL)

(21) Appl. No.: 17/438,958

(22) PCT Filed: Mar. 16, 2020
(86) PCT No.: PCT/IB2020/052402

§ 371 Date: Sep. 14, 2021

Related U.S. Application Data
(60) Provisional application No. 62/822,927, filed on Mar. 24, 2019.
(60) Provisional application No. 62/822,925, filed on Mar. 24, 2019.

(51) Int. Cl. A61K 31/352; A61K 9/16; A61K 9/00; A61K 31/05; A61K 31/192; A24B 15/167; A24B 15/28

(52) U.S. Cl. CPC A61K 31/352; A61K 9/1652; A61K 9/0075; A61K 31/05; A24B 15/285; A61K 9/1664; A24B 15/167; A24B 15/283; A61K 31/192

(57) ABSTRACT

Provided is a solid homogeneous composition comprising at least one cannabinoid and a carrier having at least two of: (i) a total cannabinoid concentration greater than 5%; (ii) being in comminuted particle form, wherein at least 70 wt % of said particles are of size greater than 0.3 millimeter and less than 4.2 millimeters; (iii) comprising less than 20 wt % water; (iv) said carrier comprising cellulose; (v) said carrier comprises less than 50% by weight cannabis or hemp non-cannabinoid plant material, wherein said composition is suitable for providing cannabinoid via inhalation. Further provided are products comprising the composition, methods of use and methods of preparation thereof.

### BACKGROUND OF THE INVENTION

Cannabidiol (CBD) has been found to be useful in treating multiple indications and symptoms.

Cannabis is a complex plant comprising over 400 chemical entities of which more than 60 are cannabinoid compounds, some of which have opposing pharmacological effects. In the naturally occurring Cannabis plant, cannabinoids are synthesized and accumulated as cannabinoid acids (e.g. tetrahydrocannabinolic acid (THCa) and cannabidiolic acid (CBDA). When the herbal product is dried, stored and heated, the acids decarboxylize into their active forms, such as tetrahydrocannabinol (THC) and Cannabidiol (CBD) [De Meijer et al. 2003; The inheritance of chemical phenotype in Cannabis sativa L. Genetics 63: 335-346].

Inhalation is in some instances considered to be the preferred method for administration of cannabis due to the short onset time and high bioavailability obtained. Inhalation may be achieved either by smoking a cannabis plant material and inhaling the smoke; or by introducing a cannabis material into a vaporizer in which the cannabis material is heated, such that vapors are generated which are then inhaled.

An increasing proportion of the population uses vaporizers for cannabis inhalation. Vaporizers are preferred over smoking for minimization of undesired smoke and burning co-products. Current solutions involve vaporizing cannabinoids in particular solvent and vaporizing dried cannabis flowers.

Vaporizing employs elevated temperatures, e.g. 200 degrees Celsius. In fact, higher temperatures would lead to higher yield. However, at these high temperatures, on using cannabis solutions, carrier vapors co-evaporate with the cannabinoids and lead to inhalation of compounds such as glycol. When a plant material is vaporized, some charring of that material occurs and degradation products are liberated and co-inhaled with the cannabinoids. Additionally, vaporizers become contaminated with ash and require frequent cleaning.

The cannabis plant material used for inhalation is typically dried and cured cannabis flowers. THC-rich strains are available wherein tetrahydrocannabinol (THC) concentration is high, e.g. greater than 20% or 25% and a THC:CBD ratio is greater than 1; and CBD-rich strains wherein CBD concentration is typically less than 15%, but in which a CBD:THC weight/weight ratio is high, e.g. about 20:1. There is a need for inhalation products of high CBD concentration.

Use of vaporizers for inhalation of cannabis material requires a cannabis form that is suitable for even heating. CBD-rich cannabis material has a number of disadvantages: (i) It contains other ingredient, such as THC, which may be undesirable, particularly for administration to children; (ii) It is difficult to introduce into the vaporizer, particularly when handled by children or old people; (iii) It leaves ash in the vaporizer, which is sometimes difficult to clean. There are known methods for isolating CBD from cannabis/hemp, generating almost pure CBD crystals. Such crystals are not suitable for use in vaporizers.

Hence, there is a need for vaporizer-suitable compositions that are devoid of at least some of the limitations of the prior art, and which are preferably easy to handle and utilize pure CBD.

### SUMMARY OF THE INVENTION

According to an aspect of some embodiments of the present invention, provided is a solid homogeneous composition in the form of particles, each of said particles comprising a carrier and at least one cannabinoid, which composition is characterized by having at least two of the following characteristics (i) to (v):

(i) a total cannabinoid concentration greater than 5%;
(ii) at least 70 wt % of said particles are of a size greater than 0.3 millimeter and less than 4.2 millimeters;
(iii) comprising less than 20 wt % water;
(iv) said carrier comprising cellulose;
(v) said carrier comprises less than 50% by weight cannabis hemp non-cannabinoid plant material or hemp non-cannabinoid plant material,
wherein said composition is suitable for providing cannabinoid via inhalation.

According to a further aspect of some embodiments of the present invention, provided is a product comprising the homogeneous composition as disclosed herein in a form selected from the group consisting of cigarettes, vaporizer feed and vaporizer cartridges.

According to a further aspect of some embodiments of the present invention, provided is a method for administering cannabidiol and/or cannabidiolic acid to a subject, comprising providing to said subject a vapor and/or aerosol for inhalation, whereby said vapor and/or aerosol is formed by maintaining a homogeneous composition as disclosed herein at a temperature of at least 150 degrees Celsius for at least 10 seconds.

According to an aspect of some embodiments of the present invention, provided is a method for producing the homogeneous composition as disclosed herein, comprising (i) providing at least one carrier in the form of a particle; (ii) providing a solution comprising at least one cannabinoid in a solvent, wherein the total concentration of cannabinoid in the solution is at least 2% by weight and wherein the boiling point of said solvent at 0.1 bar is less than 50 degrees Celsius; (iii) evenly applying the solution to the at least one carrier; and (iv) removing at least a fraction of the solvent from the solution applied to the carrier, thereby producing at least one said particle comprising a carrier and a cannabinoid-comprising coating layer on said carrier.

### DETAILED DESCRIPTION OF THE INVENTION

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention.

As used in the description of the invention and the appended claims, the singular forms “a,” “an,” and “the” are intended to include the plural forms as well, unless the context clearly indicates otherwise.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

As used herein, when a numerical value is preceded by the term “about”, the term “about” is intended to indicate +/−10% of that value.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

According to an aspect of some embodiments of the present invention, provided is a solid homogeneous composition in the form of particles, each of said particles comprising a carrier and at least one cannabinoid, which composition is characterized by having at least two of the following characteristics (i) to (v)

(i) a total cannabinoid concentration greater than 5%;
(ii) at least 70 wt % of said particles are of a size greater than 0.3 millimeter and less than 4.2 millimeters;
(iii) comprising less than 20 wt % water;
(iv) said carrier comprising cellulose;
(v) said carrier comprises less than 50% by weight cannabis non-cannabinoid plant material or hemp non-cannabinoid plant material, wherein said composition is suitable for providing cannabinoid via inhalation.

As used herein, the term “homogeneous composition” refers to a composition that has the same proportion of components throughout any given sample.

In embodiments the homogeneous composition comprises a mixture having the same proportion of ingredients throughout any given sample, although the individual particles themselves are heterogeneous.

According to an embodiment, the homogeneous composition, when separated into at least a 10 gram sample, contains a cannabidiol and cannabidiolic acid combined concentration in a 1 gram fraction of said at least 10 gram sample within 15% of the cannabidiol and cannabidiolic acid combined concentration in a separate 1 gram fraction of said at least 10 gram sample;

According to an embodiment, said homogeneous composition is characterized by having at least two of said characteristics (i) to (v), at least three, at least four, at least five of said characteristics (i) to (v) or all five of said characteristics.

According to an embodiment, said at least one cannabinoid comprises cannabidiol (CBD) and/or cannabidiolic acid (CBDa).

According to an embodiment, said total cannabinoid concentration is greater than 5% wt, greater than 10% wt greater than 15% wt, greater than 20% and greater than 25%.

According to an embodiment, at least 70 wt %, at least 75%, at least 80%, at least 85%, at least 90%, at least 95% or even 100% of said particles are of a size greater than 0.3 millimeter and less than 4.2 millimeters.

According to an embodiment, at least 70% of said particles are of a size greater than 0.3 millimeter, greater than 0.5 millimeter, greater than 1.0 millimeter, greater than 1.5 millimeter, greater than 2.0 millimeter, greater than 2.5 millimeter, greater than 3.0 millimeter, or greater than 4.0 millimeter.

According to an embodiment, at least 70% of said particles are of a size less than 4.0 millimeter, less than 3.5 millimeter, less than 3.0 millimeter, less than 2.5 millimeter, less than 2.0 millimeter, less than 1.5 millimeter, less than 1.0 millimeter or less than 0.5 millimeter.

According to an embodiment, at least 70% of said particles are of a size in the range of 0.3-4.2 millimeter, 0.5-4.0 millimeter, 0.5-3.5 millimeter, 1.0-3.0 millimeter, 1.5-2.5 millimeter or 2.0-2.5 millimeter.

According to an embodiment, said homogeneous composition comprises less than 20% water, less than 15% water, less than 10% water, less than 5% water, less than 2% water or even less than 1% water.

According to an embodiment, said carrier comprises less than 50%, less than 45%, less than 40%, less than 35%, less than 30%, less than 25%, less than 20%, less than 15%, less than 10% or even less than 5% by weight cannabis or hemp non-cannabinoid plant material. According to an embodiment, said carrier comprises at least 50%, at least 55%, at least 60%, at least 65%, at least 70%, at least 75%, at least 80%, at least 85%, at least 90%, or at least 95% by weight inorganic material and/or active carbon. According to an embodiment, said inorganic material is selected from the group consisting of ceramic material, silica, zeolite, and combinations

According to an embodiment, said homogenous composition further comprises at least one additional cannabinoid in an acid form and/or in a decarboxylated form.

According to an embodiment, said homogenous composition comprises

(i) cannabidiol (CBD) and/or cannabidiolic acid (CBDa) and
(ii) tetrahydrocannabinol (THC) and/or tetrahydrocannabinolic acid (THCa),
wherein the weight per weight ratio between a total weight of cannabidiol and/or cannabidiolic acid, and a total weight of tetrahydrocannabinol and/or tetrahydrocannabinolic acid of at least 10:1.

According to an embodiment, said homogeneous composition comprises at least one terpene. According to an embodiment, said homogeneous composition comprises at least one flavonoid. According to an embodiment, said homogeneous composition further comprises a surfactant.

According to an embodiment, said carrier is selected from the group consisting of a plant material, an inorganic material, active carbon and combinations thereof.

According to an embodiment, said plant material is selected from the group consisting of cannabis plant material, extracted cannabis plant material, hemp plant material, extracted hemp plant material, herb plant material, fruit material hemicellulosic material and combinations thereof.

According to an embodiment, said cannabis plant material carrier comprises cannabis flowers, optionally dried, optionally in a comminuted form, e.g. ground and optionally extracted, at least partially.

According to an embodiment, said hemp plant material carrier comprises hemp flowers, optionally dried, optionally in a comminuted form, e.g. ground and optionally extracted, at least partially.

According to an embodiment, said herb plant material carrier is selected from herbs used in herbal medicine and herbs used for the manufacture of essential oil.

According to an embodiment, said fruit carrier comprises fruits, optionally in dried form, optionally comminuted, e.g. ground.

According to an embodiment, said plant material is selected from the group consisting of coffee, cocoa, and cinnamon.

According to an embodiment, said cellulosic carrier comprises at least one of cigarette paper, cigarette filter and dried algae.

According to an embodiment, said inorganic material is selected from the group consisting of ceramic material, silica, zeolite, and combinations thereof.

According to an embodiment, the homogeneous composition further comprises an outer coating layer.

According to an embodiment, said carrier comprises particles having a surface area of more than 50 square meters per gram.

According to an embodiment, said carrier comprises particles made out of ceramic material, silica, zeolite, and other inorganic matter, and/or active carbon, e.g. beads or granulated matter.

According to an embodiment, said carrier comprises active carbon and/or an inorganic matter and said composition is characterized by having a cannabidiol and cannabidiolic acid combined concentration greater than 5% by weight, greater than 20%, greater than 25% or greater than 30%.

According to an embodiment, said carrier comprises a hydrophobic surface.

According to an embodiment, said homogeneous composition comprises micro-capsules containing at least one of cannabidiol (CBD) and cannabidiolic acid (CBDa), optionally at least one of tetrahydrocannabinol (THC) and tetrahydrocannabinolic acid (THCa) and optionally other cannabinoids and/or terpenes and said carrier forms at least a fraction of the coating of said capsules.

According to an embodiment, said composition is characterized by having a cannabidiol and cannabidiolic acid combined concentration greater than 5% by weight, greater than 10%, greater than 15%, greater than 20%, greater than 25% or greater than 30%.

According to an embodiment, said composition further comprises at least one of tetrahydrocannabinol and tetrahydrocannabinolic acid and is characterized by having weight per weight ratio between cannabidiol and cannabidiolic acid combined, and tetrahydrocannabinol and tetrahydrocannabinolic acid combined, of at least 10:1, at least 15:1, at least 20:1, at least 25:1, at least 30:1 or at least 35:1.

According to an embodiment, said composition is characterized by being in comminuted particle form, wherein at least 70 wt % of the particles are of a size greater than 0.3 millimeter and less than 4.2 millimeters, at least 75%, at least 80%, at least 85%, at least 90% or at least 95%. According to an embodiment, said particles are of a size greater than 0.5 millimeters, greater than 0.7, greater than 0.9, greater than 1.1 or greater than 1.3 millimeter. According to an embodiment, said particles are of a size smaller than 4 millimeters, smaller than 3.5 or smaller than 3 millimeters.

According to an embodiment, said composition is characterized by comprising at least 1 wt % moisture and less than 20 wt % moisture, at least 2%, at least 3%, at least 4%, at least 5%, at least 6%, at least 7%, at least 8%, at least 9% or at least 10%.

According to various embodiments, said composition is characterized by said carrier comprising cellulose, optionally at least 20% wt cellulose, at least 30%, at least 40%, at least 50%, at least 60%, at least 70% or at least 80%.

According to an embodiment, said composition is characterized in that, when separated into at least a 10 gram sample, contains a cannabidiol and cannabidiolic acid combined concentration in a 1 gram fraction of the at least 10 gram sample within 15% of the cannabidiol and cannabidiolic acid combined concentration in a separate 1 gram fraction of said at least 10 gram sample, within 10% or within 5%.

According to an embodiment, said composition is suitable for administering at least one of cannabidiol and cannabidiolic acid to a subject. According to an embodiment, said composition is suitable for providing cannabidiol via inhalation, e.g. via a cigarette or a vaporizer. According to an embodiment, said composition is suitable for generating vapors of at least one of cannabidiol and cannabidiolic acid on heating thereof.

According to a further aspect of some embodiments of the present invention, provided is a product comprising the solid homogeneous composition as disclosed herein and optionally other ingredients, wherein said product is in a form selected from the group consisting of cigarettes, vaporizer feed and vaporizer cartridges.

According to an aspect of some embodiments of the present invention, provided is a method for administering cannabidiol and/or cannabidiolic acid to a subject, comprising providing to said subject a vapor and/or aerosol for inhalation, whereby said vapor and/or aerosol is formed by maintaining a homogeneous composition as disclosed herein at a temperature of at least 150 degrees Celsius for at least 10 seconds.

According to an aspect of some embodiments of the present invention, provided is a method for administering cannabidiol and/or cannabidiolic acid to a subject, comprising providing to said subject a product as disclosed herein, wherein said product provides to said subject a vapor and/or aerosol for inhalation, whereby said vapor and/or aerosol is formed by maintaining said composition at a temperature of at least 150 degrees Celsius for at least 10 second.

According to an aspect of some embodiments of the present invention, provided is a method for producing the homogeneous composition as disclosed herein, comprising (i) providing at least one carrier in the form of a particle; (ii) providing a solution comprising at least one cannabinoid in a solvent, wherein the total concentration of cannabinoid in the solution is at least 2% by weight and wherein the boiling point of said solvent at 0.1 bar is less than 50 degrees Celsius; (iii) evenly applying said solution to said at least one carrier; and (iv) removing at least a fraction of the solvent from said solution applied to said carrier, thereby producing at least one said particle comprising a carrier and a cannabinoid-comprising coating layer on said carrier.

According to some embodiments, said total concentration of cannabinoid in the solution is at least 2%, at least 5%, at least 10%, at least 15%, at least 20%, at least 25%, at least 30%, at least 35%, at least 40%, at least 45% or up to 50% by weight.

According to an embodiment, said solvent is less hydrophobic than 70% ethanol in water.

According to an embodiment, said carrier is selected from the group consisting of cannabis plant material, extracted cannabis plant material, hemp plant material, extracted hemp plant material, herb plant material, fruits, cellulosic material, hemicellulosic material, ceramic particles and combinations thereof.

According to an embodiment, said solvent comprises ethanol.

According to an embodiment, said providing a solution of said cannabinoid in a solvent comprises extracting a plant material comprising said cannabinoid with said solvent to form an extract comprising said at least one of cannabidiol and cannabidiolic acid and said solvent.

According to an embodiment, said method further comprises isolating said cannabinoid from said extract and dissolving said isolated at least one of cannabidiol and cannabidiolic acid in said solvent.

### EXAMPLES

Examples of inhalation products are presented in the following table.

| | | Carrier | Moisture | CBDa | THCa | Total terpenes |
|---|---|---|---|---|---|---|
| | | content | content | concentration | concentration | concentration |
| Example | Carrier | (% wt) | (% wt) | (% wt) | (% wt) | (% wt) |
| 1 | Ground dried cannabis | 55 | 11 | 30 | 1 | 1.5 |
| | flowers | | | | | |
| 2 | Ground dried hemp | 60 | 10 | 25 | 1 | 1 |
| | flowers | | | | | |
| 3 | Porous ceramic particles | 80 | 0 | 15 | <0.5 | 1 |
| 4 | Silica with Hydrophobic | 85 | 0 | 10 | <0.5 | <0.5 |
| | surface | | | | | |
| 5 | Granulated active carbon | 70 | 0 | 25 | 1 | 1 |
| 6 | Granulated coffee | 85 | 0 | 10 | <0.5 | <0.5 |
| 7 | Zeolite | 80 | | 15 | <0.5 | 1 |
| 8 | Porous ceramic particles | 70 | 0 | 15 | 10 | 1 |
| 9 | Porous ceramic particles | 75 | 0 | 20 | 4 | 0.5 |
| 10 | Porous ceramic particles | 75 | 0 | 4 | 20 | 1.5 |
| 11 | Porous ceramic particles | 70 | 0 | 1 | 25 | 0.5 |
| 12 | Silica with Hydrophobic | 80 | 0 | 10 | 10 | <0.5 |
| | surface | | | | | |
| 13 | Granulated active carbon | 70 | 0 | 25 | 1 | 1 |
| 14 | Granulated active carbon | 75 | 0 | 20 | 4 | 0.5 |

### Exemplary Producing Method

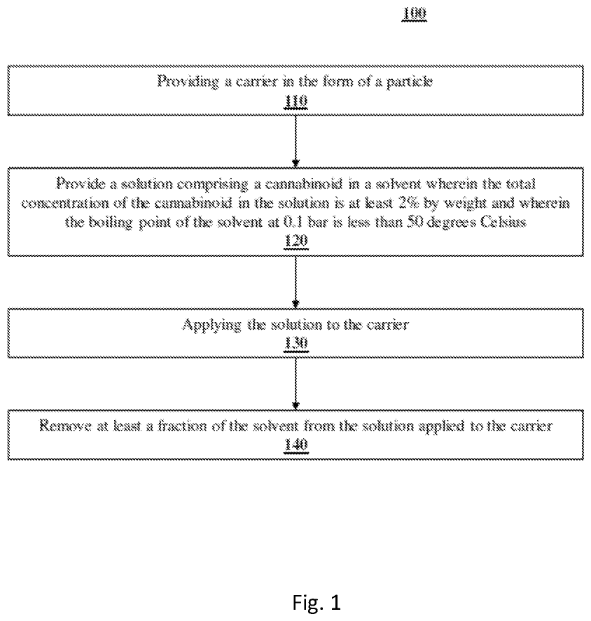

Referring now to FIG. 1, a method for producing homogeneous composition in accordance to an embodiment of the present invention, and is generally indicated as Method 100. Method 100 includes providing a carrier in the form of a particle (110); providing a solution comprising a cannabinoid in a solvent wherein the total concentration of said at least one cannabinoid in said solution is at least 2% by weight and wherein the boiling point of said solvent at 0.1 bar is less than 50 degrees Celsius (120); applying the solution to the carrier (130); and removing at least a fraction of the solvent from the solution applied to the carrier (140), thereby producing at least one said particle comprising a carrier and a cannabinoid-comprising coating layer on said carrier. According to an embodiment the carrier used in step 11o is selected from the group consisting of cannabis plant material, extracted cannabis plant material, hemp plant material, extracted hemp plant material, herb plant material, cellulosic material, hemicellulosic material, ceramic particles and combinations thereof. According to an embodiment, the solvent used in step 120 comprises ethanol.

1. A solid homogeneous composition in the form of particles, each of said particles comprising a carrier and at least one cannabinoid, which composition is characterized by having at least two of the following characteristics (i) to (v)
(i) a total cannabinoid concentration greater than 5%;
(ii) at least 70 wt % of said particles are of a size greater than 0.3 millimeter and less than 4.2 millimeters;
(iii) comprising less than 20 wt % water;
(iv) said carrier comprising cellulose;
(v) said carrier comprises less than 50% by weight cannabis non-cannabinoid plant material or hemp non-cannabinoid plant material,
wherein said composition is suitable for providing cannabinoid via inhalation.

2. The homogeneous composition of claim 1 characterized by having at least three of said characteristics (i) to (v).

3. The homogeneous composition of claim 1 characterized by having at least four of said characteristics (i) to (v).

4. The homogeneous composition of claim 1 characterized by having all five characteristics (i) to (v).

5. The homogeneous composition of claim 1, wherein said at least one cannabinoid comprises cannabidiol (CBD) and/or cannabidiolic acid (CBDa).

6. The homogeneous composition of claim 1, comprising
(i) cannabidiol (CBD) and/or cannabidiolic acid (CBDa) and
(ii) tetrahydrocannabinol (THC) and/or tetrahydrocannabinolic acid (THCa),
wherein the weight per weight ratio between a total weight of cannabidiol and/or cannabidiolic acid, and a total weight of tetrahydrocannabinol and/or tetrahydrocannabinolic acid is at least 10:1.

7. The homogeneous composition of claim 1, wherein said carrier is selected from the group consisting of a plant material, an inorganic material, active carbon and combinations thereof, wherein said plant material is optionally selected from the group consisting of cannabis plant material, extracted cannabis plant material, hemp plant material, extracted hemp plant material, herb plant material, fruit material hemicellulosic material and combinations thereof.

8. (canceled)

9. The homogeneous composition of claim 7, wherein said inorganic material is selected from the group consisting of ceramic material, silica, zeolite, and combinations thereof.

10. The homogeneous composition of claim 1, further comprising an outer coating layer.

11. The homogeneous composition of claim 1, wherein said carrier comprises particles having a surface area of more than 50 square meters per gram.

12. The homogeneous composition of claim 1, wherein said carrier comprises a hydrophobic surface.

13. The homogeneous composition of claim 1, further comprising a terpene.

14. The homogeneous composition of claim 1, further comprising a surfactant.

15. A product comprising the solid homogeneous composition of claim 1, wherein said product is in a form selected from the group consisting of cigarettes, vaporizer feed and vaporizer cartridges.

16. A method for administering cannabidiol and/or cannabidiolic acid to a subject, comprising providing to said subject a vapor and/or aerosol for inhalation, whereby said vapor and/or aerosol is formed by maintaining a homogeneous composition according to claim 1 at a temperature of at least 150 degrees Celsius for at least 10 seconds.

17. A method for administering cannabidiol and/or cannabidiolic acid to a subject, comprising providing to said subject a product according to claim 15, wherein said product provides to said subject a vapor and/or aerosol for inhalation, whereby said vapor and/or aerosol is formed by maintaining said composition at a temperature of at least 150 degrees Celsius for at least 10 seconds.

18. A method for producing the homogeneous composition of claim 1 comprising
(i) providing at least one said carrier in the form of a particle;
(ii) providing a solution comprising said at least one cannabinoid in a solvent, wherein the total concentration of said at least one cannabinoid in said solution is at least 2% by weight and wherein the boiling point of said solvent at 0.1 bar is less than 50 degrees Celsius;
(iii) evenly applying said solution to said at least one carrier; and
(iv) removing at least a fraction of the solvent from said solution applied to said carrier, thereby producing at least one said particle comprising a carrier and a cannabinoid-comprising coating layer on said carrier.

19. The method of claim 18, wherein said carrier is selected from the group consisting of cannabis plant material, extracted cannabis plant material, hemp plant material, extracted hemp plant material, herb plant material, fruits, cellulosic material, hemicellulosic material, ceramic particles and combinations thereof.

20. The method of claim 18, wherein said solvent comprises ethanol.

21. The method of claim 18, wherein said providing a solution of said cannabinoid in a solvent comprises extracting a plant material comprising said cannabinoid with said solvent to form an extract comprising said at least one of cannabidiol and cannabidiolic acid and said solvent, the method optionally further comprising isolating said cannabinoid from said extract and dissolving said isolated cannabinoid in said solvent.

22. (canceled)
